(19) United States
(12) Patent Application Publication
(10) Pub. No.: US 2024/0374312 A1
(43) Pub. Date: Nov. 14, 2024

(54) INCIDENT POWER INFERENCE FROM BUBBLE LENGTH MEASUREMENT FOR LITHOTRIPSY DEVICES

(71) Applicant: Lumenis Ltd., Yokneam Ilit (IL)

(72) Inventors: Vitaly Rondel, Hadera (IL); Asaf Granot, Lotem (IL)

(73) Assignee: Lumenis Ltd., Yokneam Ilit (IL)

(21) Appl. No.: 18/659,898

(22) Filed: May 9, 2024

Related U.S. Application Data
(60) Provisional application No. 63/501,240, filed on May 10, 2023.

(51) Int. Cl. A61B 18/26; A61B 18/00

(52) U.S. Cl. CPC A61B 18/26; A61B 2018/00511; A61B 2018/00517; A61B 2018/00642; A61B 2018/00678; A61B 2018/00684; A61B 2018/00696; A61B 2018/00785; A61B 2018/263

(57) ABSTRACT

Described herein are laser emitting medical devices and techniques implementable by laser emitting medical devices to determine a power of light emitted from a distal end of an optical fiber coupled to the medical device where the distal end of the optical fiber is disposed in a liquid environment. The devices and techniques provide to determine the power of the emitted light based on the duration or length of a bubble formed in the liquid environment by the emitted light.

### CROSS-REFERENCE TO RELATED APPLICATIONS

This application claims the benefit of U.S. Provisional Patent Application Ser. No. 63/501,240 filed on May 10, 2023, the disclosure of which is incorporated herein by reference.

### TECHNICAL FIELD

The present disclosure generally relates to surgical laser system. Particularly, but not exclusively, the present disclosure relates to surgical laser systems used in lithotripsy procedures.

### BACKGROUND

Medical lasers are used in a variety of procedures. Among several the procedures, laser energy is directed towards a target using a fiber as a conduit for the laser energy. One such procedure, to address renal calculi (e.g., kidney stones) is ureteral endoscopy, or lithotripsy. An endoscopic probe, with a camera or other sensor, is inserted into the patient's urinary tract to locate the calculi for removal. In endoscopic lithotripsy, the probe also includes an optical fiber, which conducts a laser beam to disintegrate the calculi as they are found.

An ideal lithotripsy procedure is fast, precise, and thorough. The medical practitioner minimizes the time that the endoscope is inserted, directs all the discharged laser energy into target calculi, and assures that no large stones or fragments remain. However, even with the advancements in technology, modern medical devices do not have a method to measure the laser energy (or power) delivered to the target (e.g., stone) during the procedure. A need therefore exists for a device that can measure the power of the laser energy delivered to the target, or rather the power of the laser energy that leaves the distal end of the fiber.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The present disclosure provides an endoscopic lithotripsy device and methods for such a device where the power of the laser energy exiting the distal end of the fiber can be measured. In general, the present disclosure provides for measuring the power or energy of the laser beam (e.g., a laser beam pulse, or the like) based on a bubble created by the laser beam when it exits the distal end of the fiber in the treatment environment.

In some embodiments, an endoscopic lithotripsy device can generate laser energy based on several laser types, such as, for example, Holmium or Thulium. Holmium and Thulium laser operate in a pulsed mode, where the laser emits pulses of laser energy. Conventional laser of this type measured the laser energy (e.g., energy per second, or the like) upstream to the fiber. As such, there is no real-time knowledge of the actual power applied to the target after exiting the fiber. It is to be appreciated that such devices operate in a liquid environment. That is, the target (e.g., stone, or the like) is within a liquid environment. The fiber is inserted into the environment and therefore emits laser energy from the fiber into the liquid. This creates a bubble of gaseous vapor within the liquid environment.

The present disclosure provides systems and techniques to measure a length of the bubble created by the laser energy as it exists the fiber and to determine the power emitted from the fiber based on the length of the bubble. It will be appreciated that the systems and techniques described herein provides to measure the bubble length in real-time (e.g., at points during the procedure) to provide a metric of the laser energy being delivered during the procedure.

Some embodiments can be implemented to provide a computer implemented method. The computer implemented method can comprise receiving, at a processor, an electrical signal generated by a light sensor, the electrical signal comprising an indication of a power of a light received at the light sensor, wherein the light received at the light sensor corresponds to a portion of a laser light reflected from a distal end of an optical fiber, wherein another portion of the laser light is emitted from the distal end of the optical fiber into a liquid environment; determining, at the processor, a duration of a bubble formed in the liquid environment based on the electrical signal; and determining, at the processor, a power of the portion of the laser light emitted from the distal end of the optical fiber into the liquid environment based on the duration.

In further embodiments, the computer implemented method can comprise identifying, by the processor, a reference in a lookup table corresponding to the duration, wherein the lookup table is stored in a memory coupled to the processor, and wherein the lookup table correlates a duration of a bubble with a power of emitted light; and determining the power of the portion of the laser light emitted from the distal end of the optical fiber based on the reference.

In further embodiments, the computer implemented method can comprise executing, by the processor, a machine learning (ML) model to generate an inference of the power of the portion of the laser light emitted from the distal end of the optical fiber, wherein the machine learning model is executed with at least the duration of the bubble as an input.

In further embodiments of the computer implemented, the optical fiber is coupled to a laser console comprising the processor.

In further embodiments of the computer implemented method, the laser console comprises a lasing system comprising: the light sensor; a laser source arranged to generate the laser light; and a beam splitter arranged to direct a portion of the laser light from the laser source to the optical fiber and arranged to direct the laser light reflected from the distal end of the optical fiber to the light sensor.

In further embodiments of the computer implemented method, the laser source comprising either a Holmium based lasing medium or a Thulium based lasing medium.

In further embodiments of the computer implemented method, the laser console further comprises an optical head comprising at least a lens arranged to couple the laser light to the optical fiber.

In further embodiments, the computer implemented method can comprise sending a control signal to the laser source to cause the laser source to generate the laser light having one or more characteristics, wherein the one or more characteristics are based in part on the power of the portion of the laser light emitted from the distal end of the optical fiber into the liquid environment.

In further embodiments, the computer implemented method can comprise receiving, at the processor, a second electrical signal generated by a second light sensor, the second electrical signal comprising an indication of a power of the laser light; and determining, at the processor, the duration of the bubble formed in the liquid environment based on the electrical signal and the second electrical signal.

In further embodiments of the computer implemented method, the laser console comprises the second light sensor and wherein the beam splitter is further arranged to direct a portion of the laser light from the laser source to the second light sensor.

In further embodiments of the computer implemented method, the optical fiber is arranged to be inserted through a working channel of a ureteroscope.

In further embodiments of the computer implemented method, the duration of the bubble is a length of the bubble.

In further embodiments of the computer implemented method, the duration of the bubble is a temporal duration of the bubble.

Some embodiments can be implemented to provide an apparatus for a laser medical device. The apparatus can comprise a processor; and memory coupled to the processor, the memory comprising instructions, which when executed by the processor cause the processor to implement the method of any of the embodiments described herein.

Some embodiments can be implemented as a computer-readable storage device comprising instruction, which when executed by a processor of a medical laser system cause the medical laser system to implement the method of any of the embodiments described herein.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1A illustrates an example lithotripsy procedure system, in accordance with at least one embodiment.

FIG. 1B illustrates a portion of the lithotripsy procedure system of FIG. 1A in greater detail.

FIG. 2A illustrates a portion of the lithotripsy procedure system of FIG. 1A in greater detail during a portion of a treatment procedure.

FIG. 2B illustrates a portion of the lithotripsy procedure system of FIG. 1A in greater detail during another portion of a treatment procedure.

FIG. 3 illustrates a lasing system, in accordance with at least one embodiment.

FIG. 4 illustrates graph of a magnitude of laser pulse over time.

FIG. 5 illustrates computing environment, in accordance with at least one embodiment.

FIG. 6 illustrates a logic flow for a computing apparatus of a lithotripsy procedure system, in accordance with at least one embodiment.

FIG. 7 illustrates a computer-readable storage medium in accordance with at least one embodiment.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

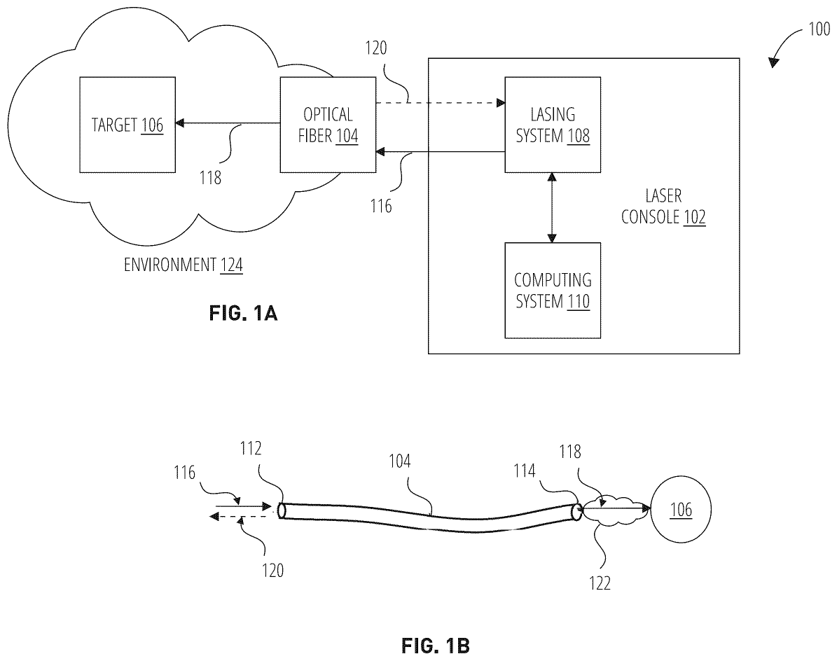

FIG. 1A and FIG. 1B show an exemplary lithotripsy procedure system 100 for determining a power (or energy) of laser light emitted from the fiber during a lithotripsy procedure, in accordance with some embodiments of the present disclosure. In some embodiments, the exemplary lithotripsy procedure system 100 comprises a laser console 102 and an optical fiber 104 configured to emit laser light (e.g., emitted laser light 118) towards a target 106. The laser console 102 can include a lasing system 108 and a computing system 110.

In some embodiments, the target 106 may be a tissue, a stone, a tumor, a cyst, and the like, within a subject, which is to be treated, ablated, or destroyed. In some embodiments, the subject may be a human being or an animal. During operation, the optical fiber 104 is coupled to the laser console 102 and inserted an environment 124 of the target (e.g., via a ureteroscope, or the like) and placed proximate to a target 106 where laser energy can be generated by the laser console 102 and directed towards the target 106 via the optical fiber 104.

As depicted more fully in FIG. 1B, the optical fiber 104 comprises a proximal end 112 and a distal end 114. The proximal end 112 is the end of the optical fiber 104 coupled to the laser console 102 and through which light beams enter while the distal end 114 is the end of the optical fiber 104 through which light beams are emitted and via which light beams can be directed onto the target 106. For example, FIG. 1B depicts laser light 116 entering the optical fiber 104 at the proximal end 112 and propagating through length of the optical fiber 104. A portion of laser light 116 exits the distal end 114 of the optical fiber 104 as emitted laser light 118 and is directed towards the target to be incident on the target 106. Furthermore, a portion of laser light 116 is reflected up the fiber by the distal end 114 as reflected laser light 120. That is, due to the interface between the distal end 114 and the environment 124 some laser light 116 will be reflected up the optical fiber 104 by the distal end 114. This is described in greater detail below.

As introduced above, often the environment 124 of the target will be a liquid environment (e.g., the bladder, kidney, or the like). As such, during operation when emitted laser light 118 is emitted from the distal end 114, a bubble 122 of gaseous vapor is formed, through which emitted laser light 118 is transmitted to be incident on target 106. Likewise

Laser light 116 can be generated by lasing system 108. Lasing system 108 may include, but is not limited to, solid-state lasers, gas lasers, diode lasers, and fiber lasers. As an illustrative example, lasing system 108 can be configured to generate laser light 116, for example, using a Holmium based lasing medium, a Thulium based lasing medium, or another type of lasing medium. Lasing system 108 can comprises optical components which may include, but are not limited to, a lasing medium, pump lights, polarizers, beam splitters, beam combiners, light detector, wavelength division multiplexers, collimators, circulators, lenses, or other such optical components arranged in various combinations to provide laser light 116.

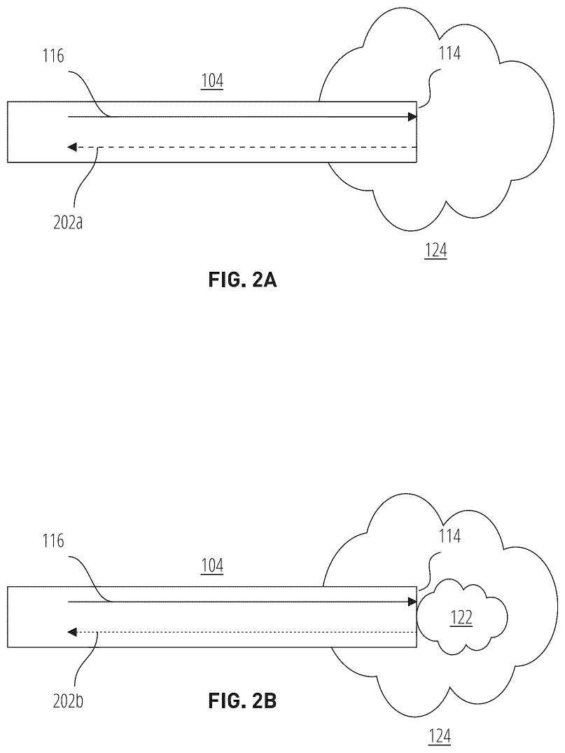

During operation, a different amount or portion of the laser light 116 will be reflected by the distal end 114 depending upon whether the bubble 122 has formed or not. For example, it is to be appreciated that, in an interface between two materials some light is reflected to the direction of the beam origin and some light is transferred through the interface. The ratio of the reflected light depends on the material optical properties of both materials in the interface. Accordingly, FIG. 2A depicts the optical fiber 104 with distal end 114 disposed in the environment 124 and laser light 116 transmitted down the optical fiber 104 and a portion of the laser light 116 reflected up the fiber as reflected laser light 202a. The magnitude or energy level of the reflected laser light 202a will depend upon the material properties of distal end 114 and environment 124, which in the present examples will be a liquid. As a specific example, Fresnel Equations could be used to determine the magnitude or power of reflected laser light 202b given the power of laser light 116 and the material properties of distal end 114 and environment 124.

When bubble 122 forms at the distal end 114 of the optical fiber 104, the interface between distal end 114 changes from distal end 114 and environment 124 (e.g., fiber/liquid) to distal end 114 and bubble 122 (e.g., fiber/gas or fiber/gas vapor). This is depicted in FIG. 2B. Given the change in the interface, the portion of the laser light 116 reflected at the interface will also change. Accordingly, reflected laser light 202b is depicted as reflected from distal end 114 of optical fiber 104. It is to be appreciated that reflected laser light 202b will have significantly more energy than reflected laser light 202a due to the fiber/gas interface versus the fiber/liquid interface. Said differently, a larger portion of laser light 116 will be reflected when the bubble 122 is present than when the bubble 122 is not present (e.g., collapses to the environment 124).

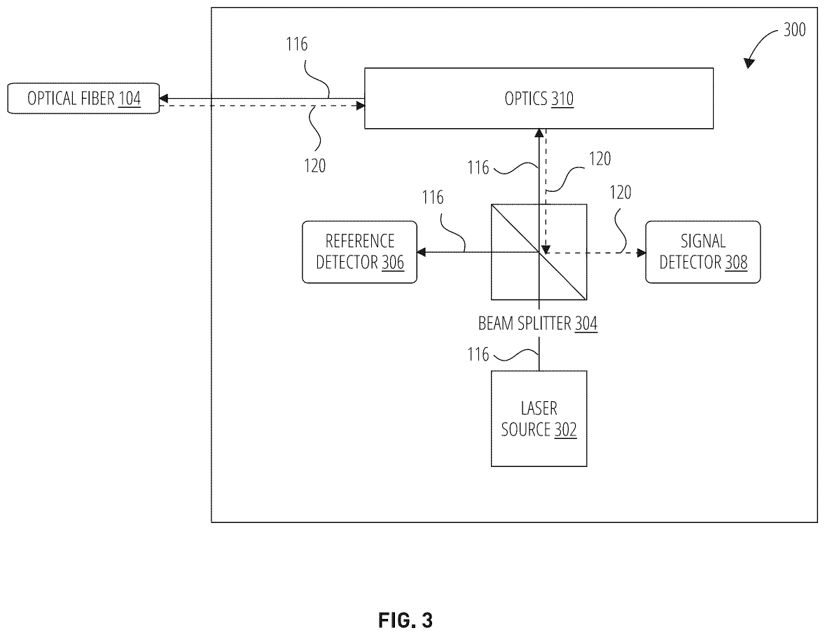

FIG. 3 illustrates an example lasing system 300, which can be implemented as lasing system 108 of lithotripsy procedure system 100 of FIG. 1A. Lasing system 300 can include a laser source 302, a beam splitter 304, a reference detector 306, a signal detector 308, and optics 310. As described above, laser source 302 can be arranged to generate laser light 116 via several different lasing mechanisms, such as, for example, using a Holmium lasing medium, using a Thulium lasing medium, or the like.

Laser light 116 can be directed to beam splitter 304, which splits the laser light 116 to direct a portion of laser light 116 to optics 310 and a portion of laser light 116 to reference detector 306. Beam splitter 304 may include any of a variety of optical components used to split incident light at a designated ratio into two separate beams. Further, beam splitter 304 may be arranged to manipulate light to be incident at a desired angle of incidence (AOI). Therefore, in many embodiments, beam splitter 304 can be primarily configured with two parameters, a ratio of separation and an AOI. The ratio of separation comprises the ratio of reflection to transmission (reflection/transmission (R/T) ratio) of the beam splitter 304. Accordingly, as used herein, if the ratio of separation for a beam splitter 304 is indicated as 1:99, it means that the beam splitter 304 splits the incident light beams in a R/T ratio of 1:99. In other words, the beam splitter 304 splits the incident light beams by changing the incident light by reflecting 1 percent and transmitting the other 99 percent. Other ratios could also be used (e.g., 50:50, 40:60, etc.) Further, as an example, if the AOI for the beam splitter 304 is indicated as 45 degrees, it means that the beam splitter 304 ensures that the light beams would be incident at an angle of 45 degrees. However, it is important to note that the present disclosure could include beam splitters of any of a variety of ratios and/or AOIs. Examples are not limited in this context. Beam splitter 304 may include, but are not limited to, polarizing beam splitters and non-polarizing beam splitters. Polarizing beam splitters may split incident light based on the S-polarization component and P-polarization component, such as, for example by reflecting the S-polarized component of light and transmitting the P-polarized component of light (or vice-versa). In some embodiments, non-polarizing beam splitters may split incident light beams based on a specific R/T ratio while maintaining the original polarization state of the incident light beams.

Optics 310 can comprise any of a variety of optical component arranged to condition and direct laser light 116 from beam splitter 304 to optical fiber 104 and direct reflected laser light 120 from optical fiber 104 to beam splitter 304. Optics 310 can include polarizers, beam combiners, collimators, circulators, lenses, etc.

From optics 310, reflected laser light 120 is directed to beam splitter 304, which reflects reflected laser light 120 to signal detector 308. Reference detector 306 and signal detector 308 can be any of a variety of light detectors. In general, such light detectors may include devices that detect and/or measure characteristics of light beams and encode the detected and/or measured characteristics in electrical signals. For example, light detectors may detect the specific type of light beams (as preconfigured), and convert the light energy associated with the detected light beams into electrical signals. These electrical signals can be communicated to a computing device (e.g., computing system 110, or the like) to determine the power of emitted laser light 118 as described herein. In general, the computing system 110 can include circuitry arranged to determine a power of emitted laser light 118 based in part on the length of the bubble 122. This is described in greater detail below.

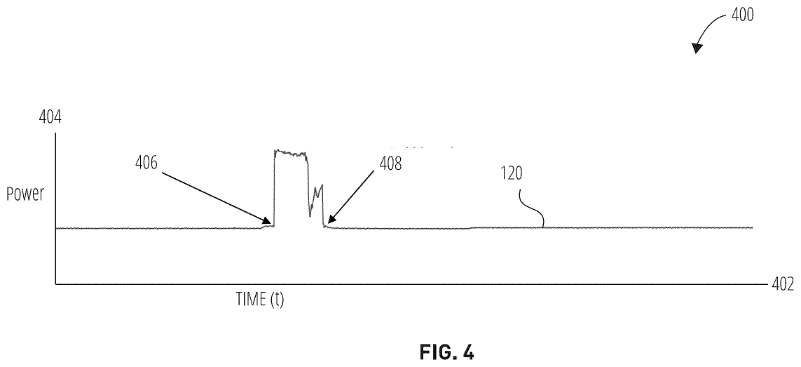

FIG. 4 illustrates a graph 400 showing the power of reflected laser light 120 over time. Time (t) is depicted on the x axis 402 while the magnitude of reflected laser light 120 is depicted on the y axis 404 as the power of reflected laser light 120 at a particular point in time. Graph 400 further depicts that the power level of reflected laser light 120 will increase when a bubble is present. For example, at the start of bubble 122 (e.g., bubble initiation 406) the power of reflected laser light 120 increases until the bubble 122 has collapsed (e.g., bubble collapse 408). Accordingly, based on the graph 400, the length, or duration, in which bubble 122 is extant can be measured. The length of bubble 122, or duration in which bubble 122 is extant, can be used to determine the power of emitted laser light 118. With some examples, a duration or time in which the magnitude or power of the reflected laser light 120 is greater than a threshold multiple (e.g., 0.75×, 1.5×, 2×, 3×, 0.75× to 2×, 0.75× to 3×, or the like) can be determined and analogized to the duration in which the bubble 122 is extant more than a selected size. For example, where the bubble 122 collapses below a certain size than the power of the reflected laser light 120 will be less than some threshold (or multiple) of the nominal power (e.g., the power when the bubble 122 is nonexistent).

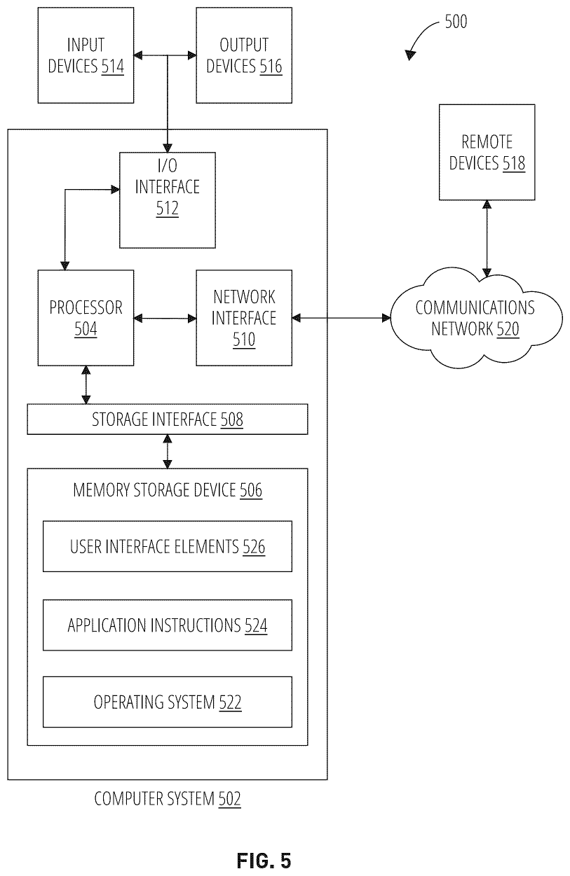

FIG. 5 is a block diagram of a computing environment 500 including a computer system 502 for implementing embodiments consistent with the present disclosure. In some embodiments, the computing environment 500, or portion thereof (e.g., the computer system 502) may comprise or be comprised in a laser system (e.g., the computing system 110 of the lithotripsy procedure system 100 can embody portions of the computing environment 500). Accordingly, in various embodiments, computer system 502 may determine a power of laser light emitted from a distal end of an optical fiber based on a duration of existence of a bubble formed by the laser light.

The computer system 502 may include a central processing unit (“CPU” or “processor”) 504. The processor 504 may include at least one data processor for executing instructions and/or program components for executing user or system-generated processes. A user may include a person, a person using a device such as those included in this disclosure, or another device. The processor 504 may include specialized processing units such as integrated system (bus) controllers, memory management control units, floating point units, graphics processing units, neural processing units, digital signal processing units, etc. The processor 504 may be disposed in communication with input devices 514 and output devices 516 via I/O interface 512. The I/O interface 512 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE), WiMAX, or the like), etc.

Using the I/O interface 512, computer system 502 may communicate with input devices 514 and output devices 516. In some embodiments, the processor 504 may be disposed in communication with a communications network 520 via a network interface 510. In various embodiments, the communications network 520 may be utilized to communicate with a remote memory storage device 506, such as for accessing look-up tables, performing updates, or utilizing external resources. The network interface 510 may communicate with the communications network 520. The network interface 510 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc.

The communications network 520 can be implemented as one of the different types of networks, such as intranet or Local Area Network (LAN), Closed Area Network (CAN) and such. The communications network 826 may either be a dedicated network or a shared network, which represents an association of the different types of networks that use a variety of protocols, for example, Hypertext Transfer Protocol (HTTP), CAN Protocol, Transmission Control Protocol/Internet Protocol (TCP/IP), Wireless Application Protocol (WAP), etc., to communicate with each other. Further, the communications network 520 may include a variety of network devices, including routers, bridges, servers, computing devices, storage devices, etcetera. In some embodiments, the processor 504 may be disposed in communication with a memory storage device 506 via a storage interface 508. The storage interface 508 may connect to memory storage device 506 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etcetera.

Furthermore, memory storage device 506 may include one or more computer-readable storage media utilized in implementing embodiments consistent with the present disclosure. Generally, a computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, non-volatile memory, hard drives, Compact Disc (CD) ROMs, Digital Video Disc (DVDs), flash drives, disks, and any other known physical storage media.

The memory storage device 506 may store a collection of program or database components, including, without limitation, an operating system 522, an application instructions 524, and a user interface elements 526. In various embodiments, the operating system 522 may facilitate resource management and operation of the computer system 502. Examples of operating systems include, without limitation, APPLE® MACINTOSH® OS X®, UNIX®, UNIX-like system distributions (E.G., BERKELEY SOFTWARE DISTRIBUTION® (BSD), FREEBSD®, NETBSD®, OPENBSD, etc.), LINUX® DISTRIBUTIONS (E.G., RED HAT®, UBUNTU®, KUBUNTU®, etc.), IBM®OS/2®, MICROSOFT® WINDOWS® (XP®, VISTA®/7/8, 10 etc.), APPLE® IOS®, GOOGLE™ ANDROID™, BLACKBERRY® OS, or the like.

The application instructions 524 may include instructions that when executed by the processor 504 cause the processor 504 to perform one or more techniques, steps, procedures, and/or methods described herein, such as to determine a power of laser light (e.g., emitted laser light 118) emitted from a distal end of an optical fiber (e.g., optical fiber 104) disposed in a liquid environment (e.g., environment 124) based on a duration of a bubble (e.g., bubble 122) formed by the laser light.

The user interface elements 526 may facilitate display, execution, interaction, manipulation, or operation of program components through textual or graphical facilities. For example, user interfaces may provide computer interaction interface elements on a display system operatively connected to the computer system 502, such as cursors, icons, checkboxes, menus, scrollers, windows, widgets, etcetera. The user interface elements 526 may be employed by application instructions 524 and/or operating system 522 to provide, for example, a user interface with which a user can interact with computer system 502. In some embodiments, the user interface elements 526 may be displayed on a display.

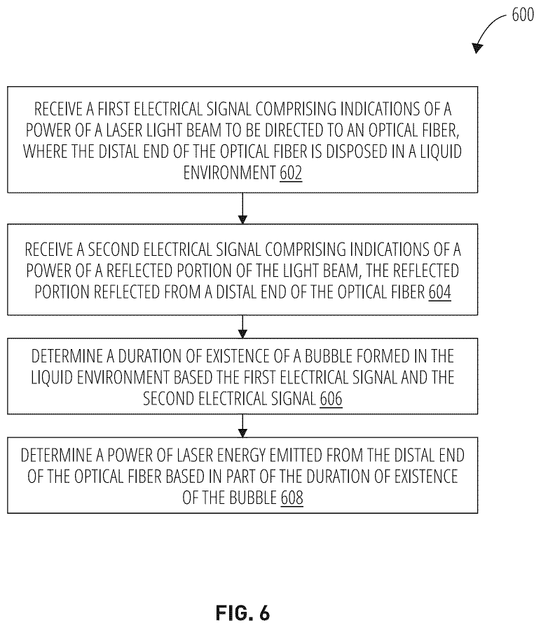

FIG. 6 illustrates a method 600 for determining a power emitted from a distal end of an optical fiber disposed in a liquid environment, according to at least one embodiment of the present disclosure. Method 600 can be implemented by a laser emitting medical device, such as, for example, lithotripsy procedure system 100. Method 600 will be described with reference to lithotripsy procedure system 100 as well as lasing system 300 and computing environment 500 for clarity of presentation. However, it is noted that method 600 could also be implemented by a laser emitting medical device different from lithotripsy procedure system 100 without departing from the scope of the disclosure.

Method 600 can begin at block 602. At block 602 “receive a first electrical signal comprising indications of a power of a laser light beam to be directed to an optical fiber, where the distal end of the optical fiber is disposed in a liquid environment” an electrical signal can be received, where the electrical signal comprises indications of a power of a laser light to be directed to an optical fiber disposed in a liquid environment. For example, processor 504 can execute application instructions 524 to receive an electrical signal from reference detector 306 comprising an indication of a power of laser light 116.

Continuing to block 604 “receive a second electrical signal comprising indications of a power of a reflected portion of the light beam, the reflected portion reflected from a distal end of the optical fiber” an electrical signal can be received, where the electrical signal comprises indications of a power of a laser light reflected from a distal end of the optical fiber. For example, processor 504 can execute application instructions 524 to receive an electrical signal from signal detector 308 comprising an indication of a power of reflected laser light 120, where reflected laser light 120 is reflected from distal end 114 of optical fiber 104 and wherein optical fiber 104 is disposed in environment 124, which is a liquid environment. Further, when reflected laser light 120 is reflected from distal end 114, emitted laser light 118 is transmitted or emitted from distal end 114 and forms bubble 122 in environment 124.

Continuing to block 606 “determine a duration of existence of a bubble formed in the liquid environment based the first electrical signal and the second electrical signal” a duration of a bubble formed by light emitted from the distal end of the optical fiber can be determined based on the electrical signal received at block 602 and the electrical signal received at block 604. In some embodiments, a ratio of the electrical signals can be generated and plotted to determine the duration of a bubble's existence. In other embodiments, a difference between the signals can be determined to determine the duration of a bubble's existence. For example, processor 504 can execute application instructions 524 to determine a duration of the bubble 122 based on signals received from reference detector 306 and signal detector 308. As a specific example, processor 504 can execute application instructions 524 to determine a time (t) in which the energy measured by signal detector 308 spikes or increases over a baseline measurement (e.g., as depicted in graph 400 of FIG. 4).

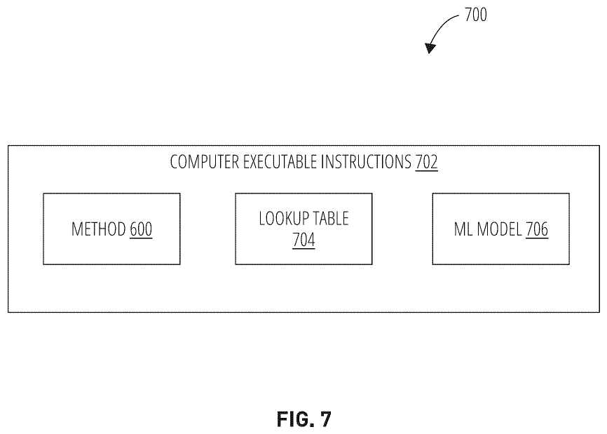

Continuing to block 608 “determine a power of laser energy emitted from the distal end of the optical fiber based in part of the duration of existence of the bubble” a power of laser energy emitted from the distal end of the optical fiber based on the duration of the bubble can be determined. For example, processor 504 can execute application instructions 524 to determine the power of emitted laser light 118 based on a formula or function where the output is the power of emitted laser light 118 and the input is the duration of the existence of bubble 122. As another example, memory storage device 506 can comprise a lookup table (e.g., see FIG. 7) correlating a duration of a bubble to emitted power and processor 504 can execute application instructions 524 to determine from the lookup table the power of emitted laser light 118 based on the determined duration of bubble 122. In still another example, memory storage device 506 can comprise (e.g., see FIG. 7) a trained machine learning (ML) model (e.g., a trained neural network, or the like) configured to receive as input a duration of a bubble and infer the power of emitted light based on the received duration. In such an example, processor 504 can be configured to execute application instructions 524 to execute the ML model to infer the power of emitted laser light 118 based on the determined duration of bubble 122.

FIG. 7 illustrates computer-readable storage medium 700. Computer-readable storage medium 700 may comprise any non-transitory computer-readable storage medium or machine-readable storage medium, such as an optical, magnetic or semiconductor storage medium. In various embodiments, computer-readable storage medium 700 may comprise an article of manufacture. In some embodiments, 700 may store computer executable instructions 702 with which circuitry (e.g., computing system 110, processor 504, or the like) can execute. For example, computer executable instructions 702 can include instructions to implement operations described with respect to method 600, computer executable instructions lookup table 704, or ML Model 706. Examples of computer-readable storage medium 700 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 702 may include any suitable type of code, such as source code, compiled code, interpreted code, executable code, static code, dynamic code, object-oriented code, visual code, and the like.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A computer implemented method, comprising:
receiving, at a processor, an electrical signal generated by a light sensor, the electrical signal comprising an indication of a power of a light received at the light sensor, wherein the light received at the light sensor corresponds to a portion of a laser light reflected from a distal end of an optical fiber, wherein another portion of the laser light is emitted from the distal end of the optical fiber into a liquid environment;
determining, at the processor, a duration of a bubble formed in the liquid environment based on the electrical signal; and
determining, at the processor, a power of the portion of the laser light emitted from the distal end of the optical fiber into the liquid environment based on the duration.

2. The computer implemented method of claim 1, comprising:
identifying, by the processor, a reference in a lookup table corresponding to the duration, wherein the lookup table is stored in a memory coupled to the processor, and wherein the lookup table correlates a duration of a bubble with a power of emitted light; and
determining the power of the portion of the laser light emitted from the distal end of the optical fiber based on the reference.

3. The computer implemented method of claim 1, comprising executing, by the processor, a machine learning (ML) model to generate an inference of the power of the portion of the laser light emitted from the distal end of the optical fiber, wherein the machine learning model is executed with at least the duration of the bubble as an input.

4. The computer implemented method of claim 1, wherein the optical fiber is coupled to a laser console comprising the processor.

5. The computer implemented method of claim 1, wherein the laser console comprises a lasing system comprising:
the light sensor;
a laser source arranged to generate the laser light; and
a beam splitter arranged to direct a portion of the laser light from the laser source to the optical fiber and arranged to direct the laser light reflected from the distal end of the optical fiber to the light sensor.

6. The computer implemented method of claim 5, wherein the laser source comprising either a Holmium based lasing medium or a Thulium based lasing medium.

7. The computer implemented method of claim 5, wherein the laser console further comprises an optical head comprising at least a lens arranged to couple the laser light to the optical fiber.

8. The computer implemented method of claim 5, comprising sending a control signal to the laser source to cause the laser source to generate the laser light having one or more characteristics, wherein the one or more characteristics are based in part on the power of the portion of the laser light emitted from the distal end of the optical fiber into the liquid environment.

9. The computer implemented method of claim 1, comprising:
receiving, at the processor, a second electrical signal generated by a second light sensor, the second electrical signal comprising an indication of a power of the laser light; and
determining, at the processor, the duration of the bubble formed in the liquid environment based on the electrical signal and the second electrical signal.

10. The computer implemented method of claim 9, wherein the laser console comprises the second light sensor and wherein the beam splitter is further arranged to direct a portion of the laser light from the laser source to the second light sensor.

11. The computer implemented method of claim 1, wherein the optical fiber is arranged to be inserted through a working channel of a ureteroscope.

12. The computer implemented method of claim 1, wherein the duration of the bubble is a time that the bubble is extant.

13. The computer implemented method of claim 1, wherein the duration of the bubble is a time that the bubble is extant greater than a threshold size.

14. An apparatus for a laser medical device, comprising:
a processor; and
memory coupled to the processor, the memory comprising instructions, which when executed by the processor cause the processor to:
receive an electrical signal generated by a light sensor, the electrical signal comprising an indication of a power of a light received at the light sensor, wherein the light received at the light sensor corresponds to a portion of a laser light reflected from a distal end of an optical fiber, wherein another portion of the laser light is emitted from the distal end of the optical fiber into a liquid environment;
determine a duration of a bubble formed in the liquid environment based on the electrical signal; and
determine a power of the portion of the laser light emitted from the distal end of the optical fiber into the liquid environment based on the duration.

15. The apparatus of claim 14, the instructions when executed by the processor further cause the processor to:
identify a reference in a lookup table corresponding to the duration, wherein the lookup table is stored in a memory coupled to the processor, and wherein the lookup table correlates a duration of a bubble with a power of emitted light; and
determine the power of the portion of the laser light emitted from the distal end of the optical fiber based on the reference.

16. The apparatus of claim 14, the instructions when executed by the processor further cause the processor to:
receive a second electrical signal generated by a second light sensor, the second electrical signal comprising an indication of a power of the laser light; and
determine the duration of the bubble formed in the liquid environment based on the electrical signal and the second electrical signal.

17. The apparatus of claim 14, wherein the optical fiber is coupled to a laser console comprising the processor and wherein the laser console comprises a lasing system comprising:
the light sensor;
a laser source arranged to generate the laser light; and
a beam splitter arranged to direct a portion of the laser light from the laser source to the optical fiber and arranged to direct the laser light reflected from the distal end of the optical fiber to the light sensor.

18. A computer-readable storage device comprising instruction, which when executed by a processor of a medical laser system cause the medical laser system to:
receive an electrical signal generated by a light sensor, the electrical signal comprising an indication of a power of a light received at the light sensor, wherein the light received at the light sensor corresponds to a portion of a laser light reflected from a distal end of an optical fiber, wherein another portion of the laser light is emitted from the distal end of the optical fiber into a liquid environment;
determine a duration of a bubble formed in the liquid environment based on the electrical signal; and
determine a power of the portion of the laser light emitted from the distal end of the optical fiber into the liquid environment based on the duration.

19. The computer-readable storage device of claim 18, the instructions when executed by the processor further cause the medical laser system to:
identify a reference in a lookup table corresponding to the duration, wherein the lookup table is stored in a memory coupled to the processor, and wherein the lookup table correlates a duration of a bubble with a power of emitted light; and
determine the power of the portion of the laser light emitted from the distal end of the optical fiber based on the reference.

20. The computer-readable storage device of claim 18, the instructions when executed by the processor further cause the medical laser system to:
receive a second electrical signal generated by a second light sensor, the second electrical signal comprising an indication of a power of the laser light; and
determine the duration of the bubble formed in the liquid environment based on the electrical signal and the second electrical signal.
